(19) United States
(12) Patent Application Publication
(10) Pub. No.: US 2006/0150984 A1
(43) Pub. Date: Jul. 13, 2006

(54) Surgical head fixation and positioning system

(76) Inventors: Joe William Ferguson, Collierville, TN (US); Robert A. Farris, Cordova, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/031,847

(22) Filed: Jan. 7, 2005

(51) Int. Cl. A61F 5/37

(52) U.S. Cl. 128/846

(57) ABSTRACT

A surgical head fixation and positioning system includes a helmet member positionable about the head of the patient and selectively conformable to the head of the patient to provide a snug fit. The system can maintain the head in position during surgery, and also allow repositioning during surgery as may be desired for placement of surgical instruments and implants.

### BACKGROUND

The head of a patient is immobilized for surgery on or near the brain, skull, cervical spine and other neurological procedures. Such immobilization may be accomplished through a number of pins and screws that are attached to the skull and engaged to a table, support, wall or other stabilizing structure adjacent to and remote from the head of the patient. In order to engage such pins and screws, it is necessary to perforate the skin and engage the skull of the patient. This results in additional time being required during the surgery and also creates locations other than the surgery location on the patient that require post-operative treatment and healing.

### SUMMARY

According to one aspect, a surgical head fixation and positioning system includes a helmet member positionable about the head of the patient and selectively conformable to the head of the patient to provide a snug fit. The system can maintain the head in position during surgery, and also allow repositioning during surgery as may be desired for placement of surgical instruments and implants.

According to another aspect, a surgical head fixation and positioning system includes a helmet member positionable about the head of the patient having removable panel portions to provide access to the cranium of the patient through the helmet-like member.

According to another aspect, a surgical head fixation and positioning system includes a helmet member positionable about the head of the patient that includes an adjustable fixture for securing the helmet member and thus the head of the patient in a desired position relative to a stabilizing structure.

According to a further aspect, a surgical head fixation and positioning system includes a helmet member positionable about the head of the patient that includes mounting members extending therefrom for securing the helmet member and thus the head of the patient in a desired position relative to a stabilizing structure.

These and other aspects will be discussed further below.

### BRIEF DESCRIPTION OF THE FIGURES

FIG. 1 is a front elevation view of a head fixation and positioning system.

FIG. 2 is a side elevation view of the system of FIG. 1.

FIG. 3 is a section view through line 3-3 of FIG. 1.

FIG. 4 is an enlarged sectional view of a portion of a helmet member comprising a portion of the system of FIG. 1 with an expansion member collapsed between portions of an engaging member.

FIG. 5 is the portion of FIG. 4 with an expansion member enlarged between portions of the engaging member.

FIG. 6 is an elevation view of the head fixation and positioning system on the head of a patient and engaged to a stabilizing structure, the patient, surgical table and stabilizing structure being shown in part.

### DESCRIPTION OF THE ILLUSTRATED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

A surgical head positioning and fixation system includes a helmet member positionable about the head of a patient. The helmet member includes a rigid outer shell and an engagement member that is releasably and fixably engageable to the head of the patient. In one embodiment, the engagement member includes a series of columnar portions and row portions that form pockets therebetween. An enlargement member having portions in the pockets can be enlarged to fixedly engage the engagement member and thus the helmet member to the head of the patient.

In another embodiment, the helmet member includes an adjustment fixture rotatable about the helmet member and securable in any one of the rotated positions relative to the helmet member. The head of the patient can be manipulated in order to allow, for example, the cervical spine to be placed in flexion, extension, compression, traction, or rotated about the central axis of the spinal column. Such repositioning of the head and cervical spine may allow or facilitate the placement of implants, instruments and other devices in a desired position relative to the cervical portion of the spine. The rotatable adjustment fixture can be rotated to a position about the helmet member that allows engagement of the adjustment fixture to a surgical table, wall, frame or other fixture to maintain the manipulated position of the head and cervical spine of the patient.

In another embodiment, the helmet member includes one or more panels removably coupled thereto. The one ore more panels can be removed for selective access to portions of the cranium of the patient for surgical procedures and placement of instruments and implants relative to the skull or brain while the remaining portion of the helmet member is fixed to the head of the patient.

In a further embodiment, the helmet member includes at least one mounting member extending therefrom. The at least one mounting member can be engaged to a surgical table, wall, frame or other stabilizing structure to maintain a positioning of the head of the patient during the surgical procedure. The helmet member can be engaged to the head of the patient while the patient is conscious. Accordingly, in the standing position the desired neutral positioning of the head and cervical portion of the spine can be imaged pre-operatively with the helmet member fixed to the patient and recorded for replication in the operating room during the surgical procedure.

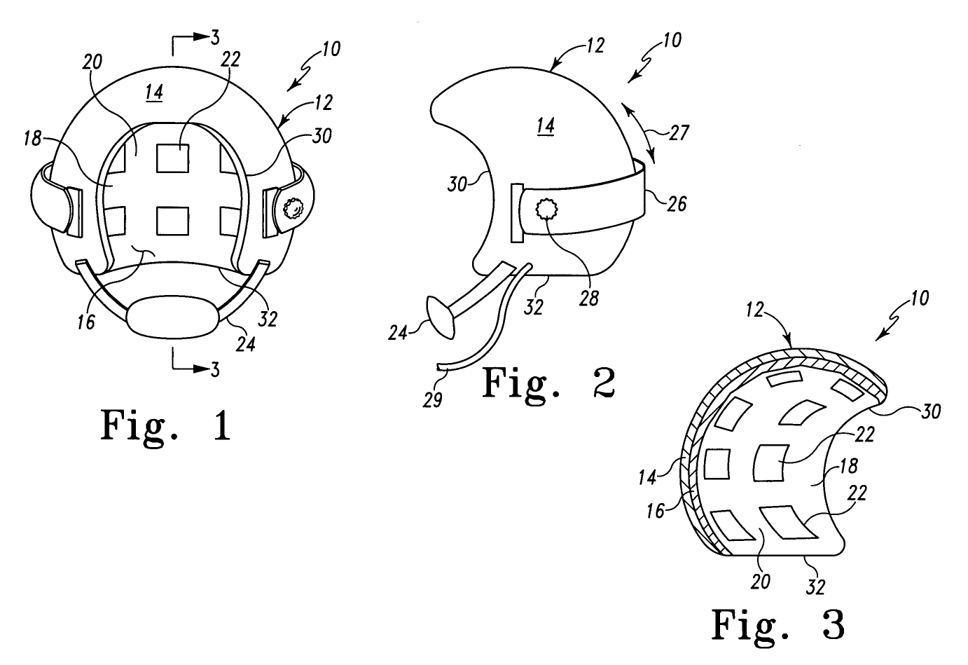

Referring to FIGS. 1-3, there is shown one embodiment of a head positioning and fixation system 10. System 10 includes a helmet member 12 forming a receptacle to receive the head of a patient with the helmet member 12 extending about the posterior and lateral portions of the head of the patient. A forward facing opening 30 is provided so that the face of the patient is exposed. Opening 30 can be shaped so the temples of the patient's head are also exposed. Alternatively, as discussed further below, helmet member 12 can be configured to cover the temples but not fixedly engage the temples. Helmet member 12 further includes a lower end opening 32 through which the neck of the patient extends when the helmet member is positioned about the head of the patient. A chin strap 24 or other member extending below the chin of the patient and between the sides of helmet member 12 may be provided adjacent lower end opening 32 across forward facing opening 30 to releasably secure the chin of the patient with helmet member 12.

Helmet member 12 includes a rigid outer shell 14 and an engagement member 16 therein that forms a lining for positioning against or adjacent the head of the patient. In the illustrated embodiment, outer shell 14 covers substantially the entire surface area of helmet member 12. Other embodiments contemplate an outer shell 14 that covers less than the entire surface area of helmet member 12. For example, outer shell 14 may comprise a series of rows and columns that align with rows and columns of the engagement member, as discussed further below.

In the illustrated embodiment, engagement member 16 can include a number of row portions 18 and a number of columnar portions 20 forming pockets 22 therebetween. At least a portion of enlargement member 34 is located in one or more of the pockets 22. In one embodiment, a portion of enlargement member 34 is located in each pocket 22. The respective portion of enlargement member 34 in a pocket 22 extends between adjacent ones of the row and columnar portions 18, 20 when enlarged to displace engagement member 16 toward the head of the patient and fixedly engage helmet member 12 thereto. In another embodiment, one or more of the pockets 22 do not include an enlargement member 34 or a portion of enlargement member 34 so that the adjacent portions of engagement member 16 are not placed in fixed engagement with the head of the patient. For example, if helmet member 12 were provided to cover the temples or eyes of the patient, then any portion of engagement member 16 in such areas would not be placed in fixed engagement with the head of the patient to avoid undue pressure on the temples or eyes.

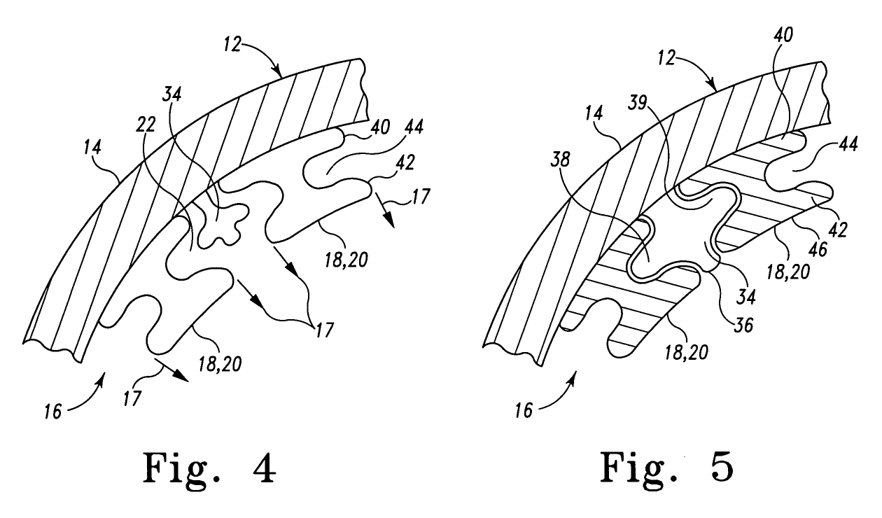

As shown in FIG. 4, the row and columnar portions 18, 20 of engagement member 16 include an I-shaped cross-section having a shell engaging portion 40, a head engaging portion 42 and a linking portion 41 therebetween. A receptacle 44 is formed between shell engaging portion 40 and head engaging portion 42. Head engaging portion 42 is movable away from shell engaging portion 40 by flexing, stretching, displacement or other action to position an engaging surface 46 in contact with the head of the patient, as indicated by arrows 17. In the illustrated embodiment, enlargement member 34 is enlargeable from its reduced size condition shown in FIG. 4 to an enlarged condition as shown in FIG. 5.

In the enlarged condition and in the illustrated embodiment, the respective portions of enlargement member 34 include arm portions 38, 39 that expand and project outwardly into adjacent receptacles 44 formed by the row and columnar portions 18, 20. The enlargement of arm portions 38, 39 into receptacles 44 moves engagement surface 46 of engagement member 16 outwardly and toward the head of the patient positioned in helmet member 12, as indicated by arrows 17 in FIG. 4. Maintaining the respective portions of enlargement member 34 in their enlarged condition fixedly engages helmet member 12 to the head of the patient with engagement member 16. Enlargement member 34 may further include a protruding portion 36 that projects between the adjacent head engaging portions 42 of row and columnar portions 18, 20 to contact the head of the patient and maintain separation between the adjacent head engaging portions 42 as enlargement member 34 is enlarged.

Other embodiments contemplate other shapes for the row and columnar portions 18, 20, including, for example, a T-shape, a C-shape, a V-shape, or any other suitable shape. The row and columnar portions 18, 20 may also include a cylindrical or other tubular shape with separate enlargement members or portions of an enlargement member housed therein. The enlargement member 34 can be shaped, for example, with outwardly extending arms and protrusions as shown, or a T-shape, spherical shape, cylindrical shape, oval shape, conical shape, frusto-conical shape, parallelepiped, star shape, diamond shape, pyramidal shape, and combinations thereof.

In still another embodiment, engagement member 16 extends completely about the inner surface of helmet member 12 and forms an inner lining that covers enlargement member 34. When enlargement member 16 is enlarged, it pushes outwardly on the engagement member 16 and places it into contact with the head of the patient. This embodiment of engagement member 16 may include row and column portions 18, 20 beneath the outer lining portion, or such row and column portions 18, 20 may be omitted.

Enlargement member 34 may include one or more portions in each of or a subset of the pockets 22. Enlargement member 34 may further include two or more separate enlargement members in respective ones of one or more pockets 22. The portions may be in the form of a bag, bladder, swellable device, or other device capable of being collapsed, deflated, un-enlarged, shrunk or otherwise assume a reduced profile configuration for positioning helmet member 12 on and off the head of the patient. The portions of enlargement member 34 may be expanded, inflated, enlarged, swelled or otherwise configured to assume an increased profile or size to fixedly engage engagement member 16 and thus helmet member 12 to the head of the patient. The portions enlargement member 34 may be enlarged, inflated and/or expanded with air, liquid, gas or other fluid or substance. The portions of enlargement member 34 may be interconnected by a network of tubes, passages or parts of adjacent enlargement member portions to allow simultaneous insertion and removal of the fluid from all the enlargement member portions. Alternatively, any one or sub-combination of enlargement member portions may be networked with one another or isolated to allow for selective enlargement of various portions of enlargement member 34. One or more leads 29 may be provided in fluid communication with one or more portions of enlargement member 34 and coupled to a fluid source (not shown) for delivery and removal of fluid to enlarge and reduce enlargement member 34.

Engagement member 16 can be in the form of a liner of shell 14 that provides a high friction engagement surface 46 positionable in fixed engagement with the skin of the head of the patient. The cross-sectional properties and/or material properties of the engagement member 16 allows portions of the engagement member 16 to move or translate toward and away from shell 14 for selective fixed engagement and release of the head of the patient. Engagement member 16 may be comprised of pads or other forms. Engagement member 16 may be comprised of a foam material made from plastic, elastomer, or other suitable material with sufficient elasticity to fixedly engage the head of the patient in response to enlargement of enlargement member 34 and allow removal of helmet member 12 when enlargement member 34 is in a reduced profile configuration. Engaging surfaces 46 can be coated with a sticky substance or other friction enhancing substance, material or surface treatment, including surface irregularities, to facilitate fixed engagement with the head of the patient and prevent or resist movement of helmet member 12 when positioned thereon.

Shell 14 can be made from any suitable material, such as plastics and polymers, metals and metal alloys, ceramics, elastomer, or composite materials, for example. Shell 14 can be made from a radiolucent material to facilitate radiographic and other imaging of the patient's head pre-operatively and during surgery. In one embodiment, shell 14 is provided with sufficient rigidity to allow rigid and fixed engagement of the helmet 12 to a surgical table, wall, frame or other stabilizing structure to prevent movement of the patient's head during surgery. Shell 14 provides a protective covering for the patient's head while allowing permanent attachment of fixation hardware that substitutes for pin fixation directly to the patient's skull.

Referring back to FIGS. 1 and 2, system 10 further includes an adjustment fixture 26 rotatably coupled to helmet member 12 and securable in any one of a number of rotated positions thereabout with a locking member 28. In the illustrated embodiment, adjustment fixture is pivotally coupled adjacent the sides of the helmet member 12 and rotatable as indicated by arrow 27. Adjustment fixture 26 can be rotated from the posterior side of helmet member 12 as shown in FIGS. 1 and 2 to an anterior side of helmet member 12 where it extends across opening 30.

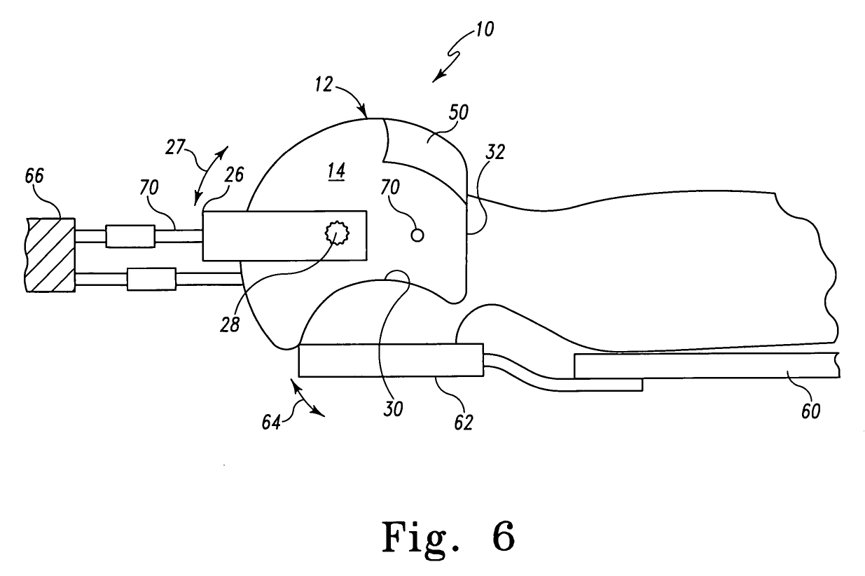

One example of an application for head positioning and fixation system 10 is shown in FIG. 6 in which a patient is lying in a prone position on surgical table 60. The face of the patient is supported in headrest 62, which may include a central opening (not shown) to accommodate eyes, nose and mouth of the patient. Headrest 62 may also be pivotally adjustable as indicated by arrows 64 to allow positioning of the cervical spine in flexion, extension and/or rotation. Other applications contemplate that surgical table supports the patient in a supine position, supports the patient lying on his or her side, or any other desired operative patient position.

Prior to surgery, the patient can be fitted with helmet member 12. The neutral positioning of the head and neck of the patient can be pre-operatively registered in a standing, supine, prone or other positioning. The registered position can be transferred into the operating room and replicated on the operating table. Helmet member 12 can be fixedly engaged to a stabilizing structure 66 with one or more mounting members 70 in the operating room while the patient remains conscious. Stabilizing structure 66 can be the operating table, wall, frame or other device which, when coupled with helmet member 12, maintains the positioning of the head of the patient relative to the surgical table.

The head of the patient can be positioned on the surgical table with helmet member 12 fixedly secured thereto. Adjustment fixture 26 can be rotated into positioned and releasably secured in the desired position with locking member 28. One or more mounting members 70 can be coupled to adjustment fixture 26 and/or directly into bores or onto bolts or hardware extending from outer shell 14 to maintain the positioning of the head of the patient on the surgical table. Mounting members 70 maintain the positioning of the head of the patient during the surgical procedure, while adjustment fixture 26 can be rotated or otherwise moved about helmet member 12 to allow repositioning of the head and neck of the patient during the procedure. Since the one or more mounting members 70 engage adjustment fixture 26 and/or rigid shell 14 rather than the patient's skull, such repositioning is facilitated as an option during the surgical procedure.

Helmet member 12 may also include one or more removable portions 50 to provide access to the skull of the patient through a window or recess in helmet member 12. Removable portion 50 may be removably coupled to helmet member 12 with screws, pins, flaps, ball-detent mechanisms, friction fit, dovetail/groove interface, snap fit, or other engaging device or means. Removable portion 50 can allow access to the skull, brain or other portion of the patient's head for performing a surgical procedure, evaluation or observation. For example, in the illustrated embodiment, removable portion 50 provides access to the occiput. Other embodiments contemplate removable portions that allow access to other portions of the skull of the patient.

While the invention has been illustrated and described in detail in the drawings and foregoing description, the same is to be considered as illustrative and not restrictive in character. All changes, modifications and equivalents that come within the spirit of the invention as defined by the following claims are desired to be protected.

What is claimed is:

1. A system for positioning a head of a patient during surgery, comprising:
a helmet member positionable about the head of the patient and configured with a forwardly facing opening for a face of the patient and a lower opening adjacent a neck of the patient when positioned on the head thereof, said helmet member including an outer shell extending about an engagement member, said engagement member defining a series of pockets at least one of which includes an enlargement member therein, said engagement member being fixedly engageable against the head of the patient upon enlargement of said enlargement member.

2. The system of claim 1, wherein said helmet member further comprises at least one panel removably coupled thereto to provide access through said helmet member to the skull of the patient during surgery.

3. The system of claim 1, wherein each of said pockets includes a portion of said enlargement member therein.

4. The system of claim 3, wherein at least two of said portions are interconnected.

5. The system of claim 1, wherein said engagement member includes a number of columnar portions and a number of row portions defining said pockets therebetween.

6. The system of claim 5, wherein said columnar portions and said row portions each include a head engaging member and a receptacle between said shell and said head engaging member, said enlargement member being enlargeable into said receptacle to move said head engaging member into contact with the head of the patient.

7. The system of claim 6, wherein said head engaging member includes a contacting surface positionable in fixed engagement with the head of the patient when said enlargement member is enlarged.

8. The system of claim 7, wherein said contacting surface includes at least one friction enhancing surface treatment.

9. The system of claim 6, wherein said columnar portions and said row portions each further include a shell engaging member opposite said head engaging member.

10. The system of claim 9, wherein said receptacle is between said shell engaging member and said head engaging member.

11. The system of claim 10, wherein at least a portion of said columnar portions and said row portions include an I-shaped cross-section.

12. The system of claim 1, further comprising a strap extendable across said forwardly facing opening and engageable to opposite sides of said outer shell when the head of the patient is positioned in said helmet member.

13. The system of claim 1, further comprising an adjustment fixture rotatably coupled to said helmet member and securable to said helmet member in a selected adjustment position.

14. The system of claim 1, further comprising at least one mounting member engageable to said adjustment fixture and a stabilizing structure, said mounting member and said stabilizing structure maintaining positioning of the head and neck of the patient when coupled with said helmet member.

15. The system of claim 14, further comprising at least one mounting member engageable between said stabilizing structure and said rigid outer shell of said helmet member.

16. The system of claim 1, further comprising at least one mounting member engageable between said stabilizing structure and said rigid outer shell of said helmet member.

17. The system of claim 1, wherein a portion of said enlargement member includes an inner chamber and is enlargeable by providing fluid to said chamber.

18. The system of claim 1, wherein when a portion of said enlargement member in said pocket is enlarged said portion includes a plurality of outwardly extending arms received in adjacent receptacles of said engagement member, said arms moving said engagement member toward the head of the patient for fixed engagement therewith.

19. The system of claim 18, wherein when said portion of said enlargement member is enlarged said portion includes a protruding portion extending between adjacent heading engaging portions of said engagement member and engaging the head of the patient.

20. The system of claim 1, wherein said outer shell is rigid.

21. A system for positioning a head of a patient during surgery, comprising:
a helmet member positionable about the head of the patient and configured with a forwardly facing opening for a face of the patient and a lower opening adjacent a neck of the patient when positioned on the head thereof, said helmet member including a hard outer shell extending about an engagement member positionable against the head of the patient to secure said helmet member in position on the head of the patient, said helmet member further comprising at least one panel removably coupled thereto to provide access through said helmet member to the skull of the patient during surgery.

22. The system of claim 21, further comprising an enlargement member including a number of portions engageable with adjacent portions of said engagement member upon enlargement of said enlargement member to position said engagement member in fixed engagement with the head of the patient.

23. The system of claim 22, further comprising an adjustment fixture coupled to said helmet member and engageable to a stabilizing structure with a mounting member to maintain the head of the patient in position during surgery.

24. The system of claim 23, wherein said adjustment fixture is articulably coupled to opposite sides of said helmet member.

25. The system of claim 21, further comprising a number of mounting members extending from said helmet member engageable to a stabilizing structure to maintain a positioning of the head of the patient during surgery.

26. The system of claim 21, wherein said engagement member includes a number of columnar portions and a number of row portions defining pockets therebetween.

27. The system of claim 26, wherein said columnar portions and said row portions each include a head engaging member and a receptacle between said shell and said head engaging member, and further comprising an enlargement member having a portion in at least one of said pockets, said portion of said enlargement member being enlargeable into said receptacle to move said head engaging member into contact with the head of the patient.

28. The system of claim 27, wherein said head engaging member includes a contacting surface positionable in contact with the head of the patient when said portion of said enlargement member is enlarged.

29. The system of claim 27, wherein said columnar portions and said row portions each further include a shell engaging member opposite said head engaging member.

30. The system of claim 29, wherein said receptacle is between said shell engaging member and said head engaging member.

31. The system of claim 30, wherein at least a portion of said columnar portions and said row portions include an I-shaped cross-section.

32. A system for positioning a head of a patient during surgery, comprising:
a helmet member positionable about the head of the patient and configured with a forwardly facing opening for a face of the patient and a lower opening adjacent a neck of the patient when positioned on the head thereof, said helmet member including a hard outer shell extending about an engagement member positionable against the head of the patient to secure said helmet member in position on the head of the patient, and further comprising a plurality of mounting members extending outwardly from said helmet member for attachment to a stabilizing structure to maintain positioning of the head and helmet member during surgery.

33. The system of claim 32, wherein said helmet member further comprises at least one panel removably coupled thereto to provide access through said helmet member to the skull of the patient.

34. The system of claim 32, wherein said mounting members are coupled at one end to said rigid shell.

35. The system of claim 32, further comprising an adjustment fixture movably coupled to said outer shell and securable to said helmet member at any one of a number of positions relative thereto.

36. The system of claim 35, wherein at least one of said plurality of mounting members is engaged to said adjustment fixture.

37. The system of claim 32, further comprising an enlargement member including a number of portions engageable with adjacent portions of said engagement member upon enlargement of said enlargement member to position said engagement member in fixed engagement with the head of the patient.

38. The system of claim 37, wherein said portions of said engagement member define receptacles for receiving outwardly extending arms of said portions of said enlargement member when enlarged.

39. The system of claim 38, wherein said adjacent portions of said engagement member include a number of columnar portions and a number of row portions defining pockets therebetween, and said portions of said enlargement member are positioned in respective pockets of said engagement member.

40. The system of claim 39, wherein said columnar portions and said row portions each include a head engaging member and said receptacles are formed between said outer shell and said head engaging member, and said enlarged portions of said enlargement member are received in said receptacles to move said head engaging member into contact with the head of the patient.

41. The system of claim 40, wherein said head engaging member includes a contacting surface positionable in fixed engagement with the head of the patient when said portion of said enlargement member is enlarged.

42. A method for stabilizing a patient's head during surgery, comprising:
positioning a helmet member about the patient's head;
enlarging an enlargement member to contact an engagement member of the helmet member with the head of the patient; and
performing a surgical procedure on at least one the head and spine of the patient.

43. The method of claim 42, wherein the helmet member includes an outer shell and said engagement member forms a liner in the outer shell.

44. The method of claim 43, wherein said outer shell is rigid.

45. The method of claim 42, wherein the helmet member includes a forwardly facing opening for the face of the patient.

46. The method of claim 42, wherein the engagement member defines a number of pockets and the enlargement member includes a portion in respective ones of the pockets.

47. The method of claim 46, wherein enlarging the enlargement member includes enlarging at least one of the portions of the enlargement member to engage adjacent portions of the engagement member to position the adjacent portions of the engagement member in contact with the head of the patient.

48. The method of claim 47, wherein the adjacent portions of the engagement member include receptacles for receiving the enlargement member.

49. The method of claim 48, wherein the adjacent portions of the engagement member are spaced from one another and enlarging the at least one portion of the enlargement member positions a protruding portion of the at least one portion of the enlargement member into contact with the head of the patient between the adjacent portions of the engagement member.

50. The method of claim 42, further comprising:
moving an adjustable fixture mounted to the helmet member to a desired position; and
coupling the adjustable fixture to a stabilizing structure remote from the helmet member.

51. The method of claim 42, further comprising:
coupling the helmet member to a stabilizing structure remote from the helmet member.

52. The method of claim 42, further comprising removing at least one panel from the helmet member to provide access to the patient's skull during surgery.

53. The method of claim 42, further comprising:
pre-operatively registering a position of the head and neck of the patient when conscious with the helmet member positioned on the head of the patient;
placing the head and neck of the patient in the registered position for surgery; and
immobilizing the head and neck of the patient for surgery by coupling the helmet member to a stabilizing structure remote from the helmet member.
